(19) United States
(12) Patent Application Publication
(10) Pub. No.: US 2023/0166079 A1
(43) Pub. Date: Jun. 1, 2023

(54) STEERABLE ELONGATE MEDICAL DEVICE

(71) Applicant: BOSTON SCIENTIFIC MEDICAL DEVICE LIMITED, Galway (IE)

(72) Inventors: Sharath Kumar G, Ramanagar District (IN); Deepak Kumar Sharma, Muzaffarnagar (IN)

(73) Assignee: BOSTON SCIENTIFIC MEDICAL DEVICE LIMITED, Galway (IE)

(21) Appl. No.: 18/071,092

(22) Filed: Nov. 29, 2022

Related U.S. Application Data
(60) Provisional application No. 63/283,837, filed on Nov. 29, 2021.

(51) Int. Cl. A61M 25/01

(52) U.S. Cl. CPC A61M 25/01; A61M 25/0136

(57) ABSTRACT

An elongate steerable medical device includes a shaft and a handle assembly that is removably securable to the shaft. The shaft includes an elongate outer member and an elongate inner member that is disposed within the elongate outer member. The handle assembly includes a proximal handle, a spool that is slidingly secured to the handle body and a collet that is removably secured to the handle body. The spool is adapted to releasable secure the elongate inner member and the collet is adapted to releasably secure the elongate outer member.

### CROSS-REFERENCE TO RELATED APPLICATIONS

This application claims the benefit of priority under 35 U.S.C. § 119 of U.S. Provisional Application No. 63/283,837, filed Nov. 29, 2021, the entire disclosure of which is hereby incorporated by reference.

### TECHNICAL FIELD

The present disclosure pertains to medical devices, and methods for manufacturing and using medical devices.

### BACKGROUND

A wide variety of medical devices have been developed for medical use, for example, for use in accessing body cavities and interacting with fluids and structures in body cavities. Some of these devices may include guidewires, catheters, pumps, motors, controllers, filters, grinders, needles, valves, and delivery devices and/or systems used for delivering such devices. These devices are manufactured by any one of a variety of different manufacturing methods and may be used according to any one of a variety of methods. Of the known medical devices and methods, each has certain advantages and disadvantages.

### SUMMARY

This disclosure provides design, material, manufacturing method, and use alternatives for medical devices. As an example, an elongate steerable medical device includes a shaft and a handle assembly that is removably securable to the shaft. The shaft includes an elongate outer member and an elongate inner member that is slidingly disposed within the elongate outer member. The handle assembly includes a handle body including a proximal handle. A spool is slidingly and releasably securable to the handle body and is adapted to be releasable securable to the elongate inner member. A knob is rotatably and releasably securable to the handle body and is adapted to be releasably securable to the elongate inner member such that rotation of the knob relative to the handle body causes rotation of the elongate inner member. A collet is removably securable to a distal end of the handle body and is adapted to releasably secure the elongate outer member relative to the collet. Movement of the spool relative to the handle body causes the elongate inner member to move relative to the elongate outer member.

Alternatively or additionally, the elongate inner member may include an elongate core wire and a sheath extending over the elongate core wire. The elongate core wire may include a proximal portion that extends proximally from the elongate outer member and an asymmetric distal portion having a semi-circular cross-sectional profile that is secured to the sheath.

Alternatively or additionally, the sheath may include one or more polymeric layers extending over the elongate core wire.

Alternatively or additionally, the sheath may further include a braided layer.

Alternatively or additionally, at least a portion of the sheath may include a laser cut hypotube.

Alternatively or additionally, the sheath may include a distal sleeve that is disposed over and secured to the asymmetric distal portion of the elongate core wire.

Alternatively or additionally, the sheath may include a proximal sleeve disposed over the proximal portion of the elongate core wire.

Alternatively or additionally, the asymmetric distal portion of the elongate core wire may include a flexibility-enhancing pattern formed therein.

Alternatively or additionally, the spool may include a first spool half and a second spool half that are adapted to releasably snap together to slidingly secure the spool to the handle body with the proximal portion of the elongate inner member extending therethrough.

Alternatively or additionally, the first spool half and the second spool half may be adapted to separate from each other and the handle body in order to allow release of the proximal portion of the elongate inner member.

Alternatively or additionally, the elongate inner member may further include a crimp sleeve secured to the proximal portion of the elongate inner member.

Alternatively or additionally, the spool may define an interior cavity adapted to accommodate the crimp sleeve therein in order to secure the proximal portion of the elongate inner member relative to the spool.

Alternatively or additionally, the knob may include a first knob half and a second knob half that are adapted to releasably snap together to rotatably secure the knob to the handle body with the proximal portion of the elongate inner member extending therethrough.

Alternatively or additionally, the first knob half and the second knob half may be adapted to separate from each other and the handle body in order to allow release of the proximal portion of the elongate inner member from the handle assembly.

Alternatively or additionally, the distal end of the handle body may include a threaded outer surface and a cavity defined by the threaded outer surface.

Alternatively or additionally, the collet may include a tapered inner collet section disposed within the cavity and an outer collet section threadedly engaged with the threaded outer surface and thus securing the elongate outer member relative to the tapered inner collet section.

Alternatively or additionally, the shaft may be a guidewire.

As another example, a handle assembly is adapted for use with a steerable elongate medical device that includes an elongate outer member and an elongate inner member disposed within the elongate outer member. The handle assembly includes a handle body including a proximal handle. The handle assembly includes a first spool half and a second spool half that are adapted to releasably snap together to form a spool slidingly secured to the handle body, the spool adapted to releasable secure the elongate inner member. A collet is removably secured to a distal end of the handle body and is adapted to releasably secure the elongate outer member device. Steering the steerable elongate medical device is enabled by moving the spool relative to the handle body.

Alternatively or additionally, the handle assembly may further include a knob including a first knob half and a second knob half that are adapted to releasably snap together, the knob rotatably secured to the handle body with the elongate inner member extending therethrough such that rotation of the knob relative to the handle body causes rotation of the elongate inner member.

As another example, a steerable guidewire assembly includes a steerable guidewire and a handle assembly. The steerable guidewire includes an elongate outer member and an elongate inner member slidingly disposed within the elongate outer member. The elongate inner member includes a proximal portion that extends proximally from the elongate outer sheath, an asymmetric distal portion having a semi-circular cross-sectional profile, and a sheath extending over the elongate inner member, the asymmetric distal portion secured to the sheath. The handle assembly includes a handle body including a proximal handle. A spool is slidingly securable to the handle body and is adapted to enable translation of the elongate inner member. A knob is rotatably securable to the handle body and is adapted to enable rotation of the elongate inner member. A collet is removably securable to a distal end of the handle body and is collet adapted to releasably secure the elongate outer member relative to the collet. Relative movement of the spool and/or the knob facilitates steering of the steerable guidewire.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of an illustrative elongate steerable medical device;

FIG. 2 is an enlarged perspective view of a portion of the illustrative elongate steerable medical device of FIG. 1;

FIG. 3 is a cross-sectional view taken along line 3-3 of FIG. 2;

FIG. 4 is a cross-sectional view taken along line 4-4 of FIG. 2;

FIG. 5 is a perspective view of a portion of the illustrative elongate steerable medical device of FIG. 1;

FIG. 6 is a perspective view of a portion of the illustrative elongate steerable medical device of FIG. 1;

FIG. 7 is a perspective view of a portion of the illustrative elongate steerable medical device of FIG. 1;

FIG. 8 is a perspective view of a portion of the illustrative elongate steerable medical device of FIG. 1;

FIG. 9 is a perspective view of a portion of the illustrative elongate steerable medical device of FIG. 1; and

FIG. 10 is an exploded perspective view of a portion of the illustrative elongate steerable medical device of FIG. 1.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

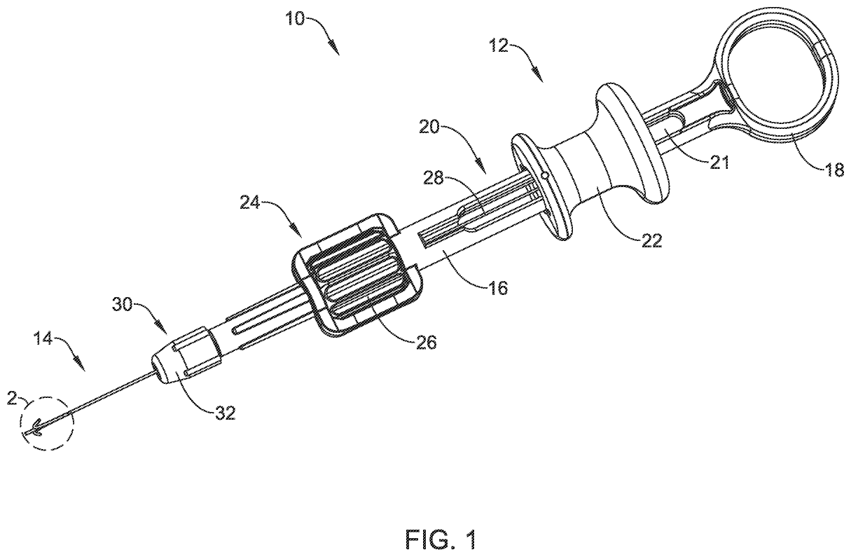

FIG. 1 is a perspective view of an illustrative steerable elongate medical device 10. The illustrative steerable elongate medical device 10 includes a handle assembly 12 and a shaft 14. In some cases, as will be discussed, the shaft 14 includes an elongate inner member 28 and an elongate outer member 40. The elongate inner member 28 may be slidingly disposed within the elongate outer member 40, for example. While the steerable elongate medical device 10 may encompass a variety of different devices, such as a variety of catheters, in some cases the shaft 14 represents a steerable guidewire. Accordingly, the invention will be illustrated with respect to a steerable guidewire. It will be appreciated that the invention is not so limited, however.

The handle assembly 12 includes a handle body 16 and a proximal handle 18 that is formed as part of the handle body 16. The handle body 16 may be formed of a variety of different materials, although in some cases the handle body 16 may be polymeric. The handle body 16 may be molded, for example, or could be 3D printed. In some cases, the handle body 16 may be formed of a polymeric material such as acrylonitrile butadiene styrene (ABS), polyvinyl chloride (PVC) and high density polyethylene (HDPE).

The handle body 16 includes a spool region 20 that accommodates a spool 22. As will be discussed, the spool 22 may be configured to translate back and forth within the spool region 20 in order to effect steering of the shaft 14. In some cases, the proximal handle 18 and the spool 22 together are configured to enable a user to hold the handle body 16 with their thumb extending through the proximal handle 18 and the spool 22 grasped between two of their fingers by virtue of a narrowed central region. It will be appreciated that this design provides ergonomic advantages. In some cases, the spool 22 is configured to be easily secured to the handle body 16 as well as to be easily removed from the handle body 16. The spool 22 may be formed of a variety of different materials. In some case, the spool 22 may be formed of a polymeric material such as acrylonitrile butadiene styrene (ABS), polyvinyl chloride (PVC) and high density polyethylene (HDPE).

The handle body 16 include a knob region 24 that accommodates a knob 26. As will be discussed, the knob 26 may be configured to rotate within the knob region 24 in order to effect rotation of the shaft 14. In some cases, the knob 26 is configured to be easily secured to the handle body 16 as well as to be easily removed from the handle body 16. An elongate core wire 28, forming part of the shaft 14, can be seen as extending through the handle body 16, through the knob 26 and into the spool 22. The knob 26 may be formed of a variety of different materials. In some case, the knob 26 may be formed of a polymeric material such as acrylonitrile butadiene styrene (ABS), polyvinyl chloride (PVC) and high density polyethylene (HDPE).

The handle body 16 includes a securement region 30 that may be used to secure the shaft 14 relative to the handle body 16. The securement region 30, which includes an outer collet member 32 as well as an inner collet member (not visible in this view). The outer collet member 32 (as well as the inner collet member) may be formed of a variety of different materials. In some case, the outer collet member 32 (as well as the inner collet member) may be formed of a polymeric material such as acrylonitrile butadiene styrene (ABS), polyvinyl chloride (PVC) and high density polyethylene (HDPE).

Like the spool 22 and the knob 26, the outer collet member 32 (and the unseen inner collet member) are easily securable to the securement region 30 and are easily removable from the securement region 30. It will be appreciated that once the shaft 14 reaches a desired position within the patient's vasculature, there is a desire to remove the handle assembly 12 from the shaft 14 such that, in the case of the shaft 14 being a guidewire, it is possible to advance other medical devices over the shaft 14.

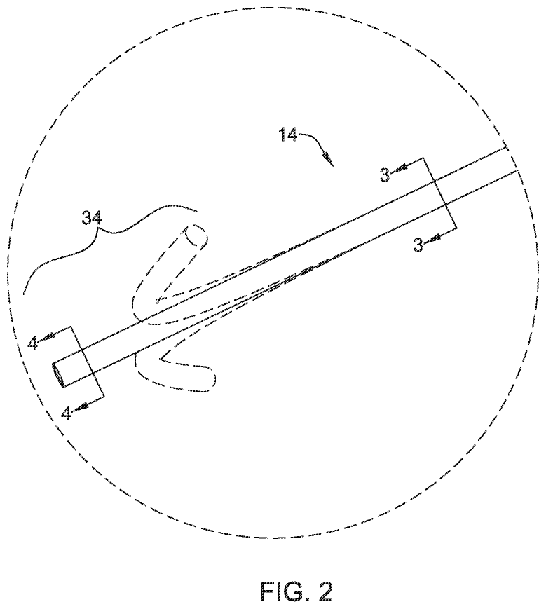

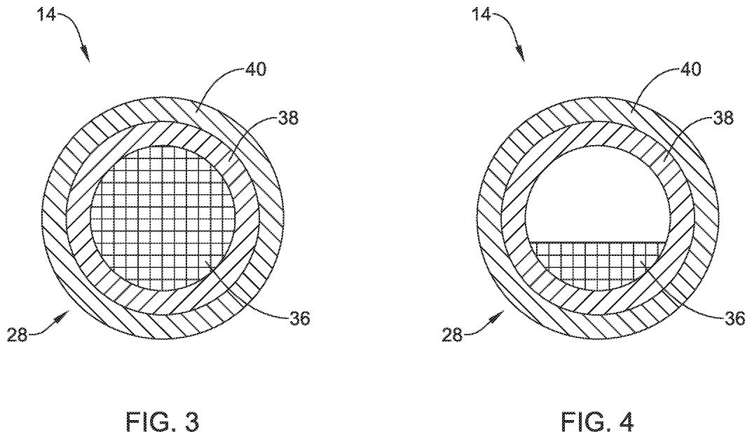

FIG. 2 is a perspective view of a distal portion of the shaft 14, including a steerable distal region 34. An unactuated configuration of the distal region 34 is shown in solid line while dashed lines are used to indicate how the distal region 34 can be actuated to turn left (or down, as illustrated) or right (or up, as illustrated), for example. FIG. 2 also indicates where the cross-sections shown in FIG. 3 (line 3-3) and FIG. 4 (line 4-4) are taken. Together, FIGS. 3 and 4 illustrate the unique configuration of the elongate inner member 28 and the elongate outer member 40 forming part of the shaft 14.

As seen in FIG. 3, the elongate inner member 28 includes an inner core member 36 and a sheath 38 that extends over the inner core member 36. In some cases, the sheath 38 may be a single polymeric layer. The sheath 38 may include two or more polymeric layers that are formed of the same polymer or different polymers. In some cases, the sheath 38 may include a braided member that is embedded within a polymeric layer or disposed between two concentric polymeric layers, for example. In some cases, the sleeve 38 may be a hypotube. In some cases, the sleeve 38 may be a hypotube having a flexibility-enhancing pattern formed within the hypotube. In some cases, the sleeve 38 may include a Nitinol™ hypotube, for example.

As seen in FIG. 4, the inner core member 36 no longer has a circular cross-sectional profile, but now has a semi-circular profile. In some cases, at least a portion of the semi-circular profile region of the inner core member 36 may be asymmetrically secured to the sleeve 38 while more proximal portions of the inner core member 36 are not secured to the sleeve 38.

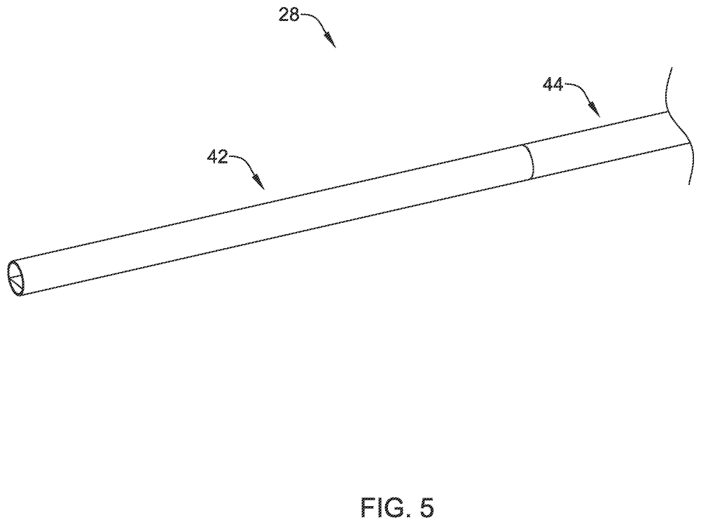

FIG. 5 is a perspective view of a distal region of the elongate inner member 28. The distal region of the elongate inner member 28 includes a distal sleeve 42 and a proximal sleeve 44. The distal sleeve 42 and the proximal sleeve 44 may be considered as being representing the sleeve 38 seen in FIGS. 3 and 4, for example. In some cases, the distal sleeve 42 may be formed of a polymeric material such as 32 durometer (32D) braided Pebax® and the proximal sleeve may be formed of a polymeric material such as 72D braided Pebax®, although other materials are contemplated.

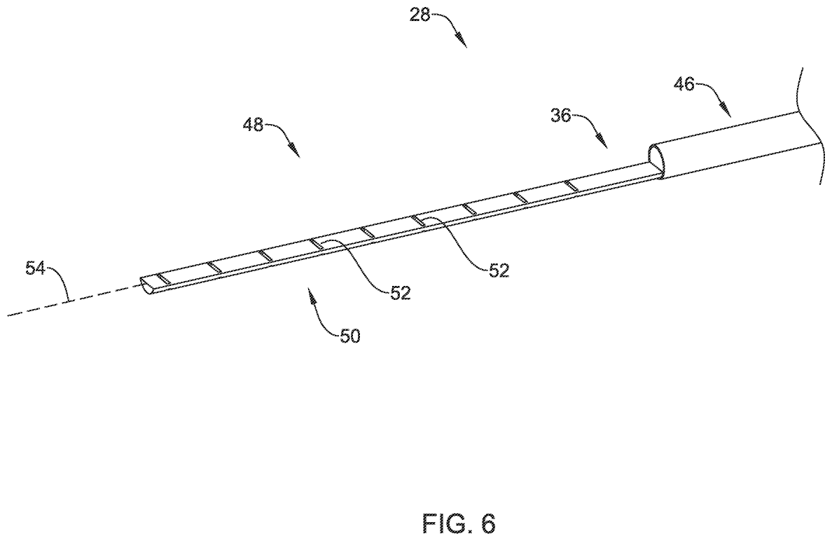

FIG. 6 is a perspective view of a distal region of the elongate inner member 28, with extraneous layers removed to expose the core member 36. As can be seen, a proximal portion 46 has a circular cross-sectional profile, as seen for example in FIG. 3 while a distal portion 48 has a semi-circular cross-sectional profile, as seen for example in FIG. 4. The distal portion 48 includes a flexibility-enhancing pattern 50 formed within the distal portion 48 in order to make the distal portion 48 more flexible. As shown, the flexibility-enhancing pattern 50 includes a number of elongate apertures 52 that extend through the core member 36. In some cases, as shown, the elongate apertures 52 may be arranged at least substantially perpendicular to a longitudinal axis 54 of the distal portion 48. The flexibility-enhancing pattern 50 may be altered to provide different flexibility and other performance parameters, for example.

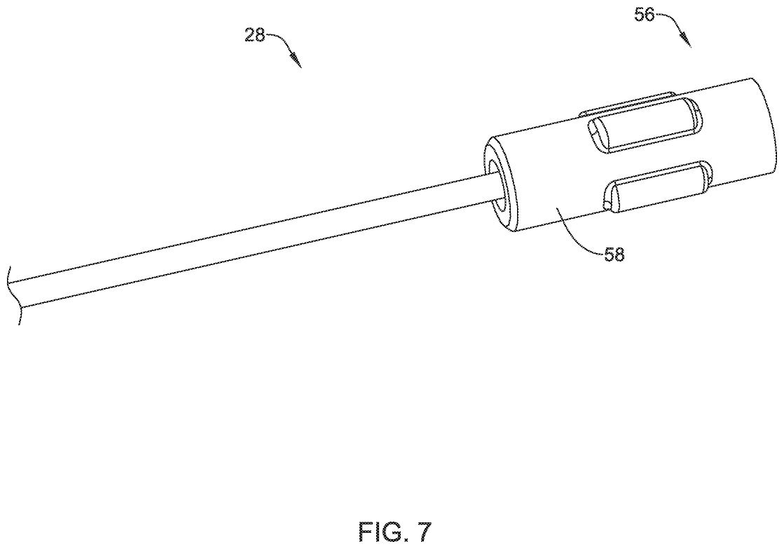

FIG. 7 is a perspective view of a proximal portion of the elongate inner member 28 including a proximal end 56. A securement crimp 58 can be seen as being attached to the proximal end 56 of the elongate inner member 28. The securement crimp 58, which can secured to the proximal end 56 of the elongate inner member 28 using any of a variety of securement techniques. In some cases, the securement crimp 58 may simply be crimped onto the proximal end 56 of the elongate inner member 28. The securement crimp 58 may be adhesively secured in place. The securement crimp 58 may be welded or soldered in place, for example. The securement crimp 58 may be formed of any suitable material such as a metallic material or a polymeric material. The securement crimp 58 may take any of a variety of different configurations, as long as the particular configuration of the securement crimp 58 serves to releasably secure the elongate inner member 28 in place relative to the spool 22 such that translation of the spool 22 causes a corresponding translation of the elongate inner member 28.

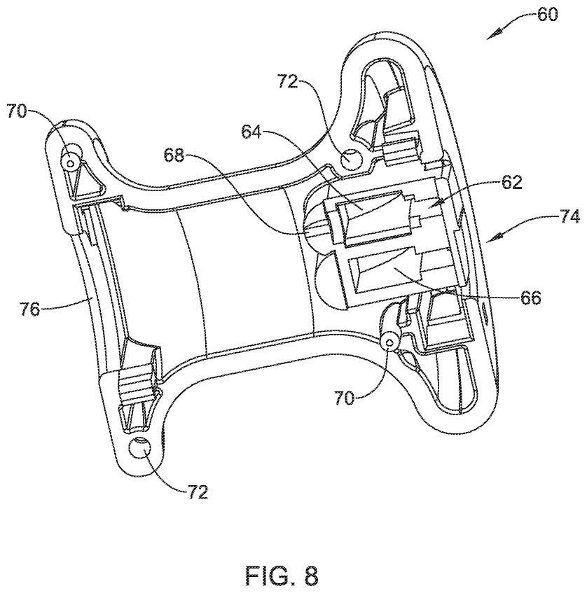

FIG. 8 is a perspective view of a spool half 60. It will be appreciated that a pair of spool halves 60 (only one is seen in FIG. 8) may be assembled together to form the spool 22. The spool half 60 includes a recess area 62 that is adapted to accommodate the securement crimp 58. In particular, a first recess 64 will cooperate with a second recess 66 (in the second spool half 60) to form the recess area 62. When two spool halves 60 are combined, one on top of the other, the first recess 64 formed in one of the spool halves 60 will align with the second recess 66 formed in the other of the two spool halves 60. The first recess 64 includes a groove 68 that is configured to accommodate the elongate inner member 28. In a similar fashion, it will be appreciated that by combining two spool halves 60, one on top of the other, that the attachment protrusions 70 of one of the spool halves 60 will align with the corresponding attachment apertures 72 formed in the other of the spool halves 60. While the attachment protrusions 70 and the corresponding attachment apertures 72 are shown as being circular, it will be appreciated that any of a variety of different complementary shapes may be used, as long as the complementary shapes provide a snap-fit with sufficient retention to hold the pair of spool halves 60 together yet permit the user to easily separate the pair of spool halves 60 when it is desired to remove the spool 22 from the handle body 16. Other forms of mechanical or frictional securements are also contemplated. The first recess 64 and the second recess 66 may take any configuration, including that shown, as long as the first recess 64 and the second recess 66 in combination accommodate the securement crimp 58 therein.

The recess area 62 may be considered as being formed within a raised portion 74 that is configured to fit into an open space 21 within the spool region 20 (FIG. 1). The spool half 60 also includes a large aperture 76 that is configured to fit around a periphery of the spool region 20. The spool half 60 may be injected molded, for example, or could be 3D printed.

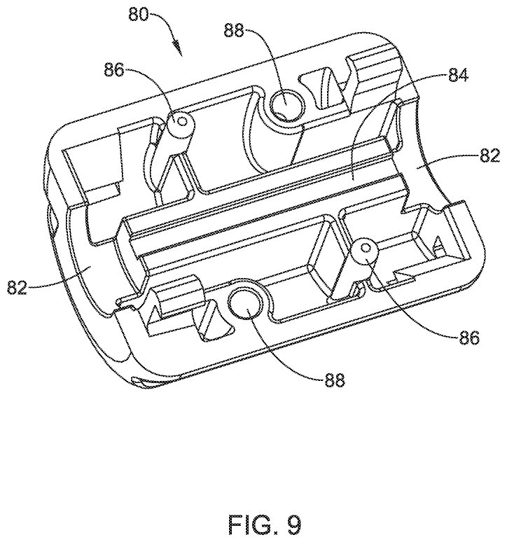

FIG. 9 is a perspective view of a knob half 80. It will be appreciated that a pair of knob halves 80 (only one is seen in FIG. 9) may be assembled together to form the knob 26. The knob half 80 includes a pair of curved rotation surfaces 82 that cooperate with the knob region 24 of the handle body 16 in order to allow the knob 26 to rotate relative to the handle body 16. The knob half 80 includes a securement region 84 that, together with the securement region 84 formed in a second knob half 80, serves to compressively secure the elongate inner member 28 relative to the knob 26 when a pair of knob halves 80 are secured together, one on top of the other.

The knob half 80 includes a pair of attachment protrusions 86 and a pair of attachment apertures 88. While the attachment protrusions 86 and the corresponding attachment apertures 88 are shown as being circular, it will be appreciated that any of a variety of different complementary shapes may be used, as long as the complementary shapes provide a snap-fit with sufficient retention to hold the pair of knob halves 80 together yet permit the user to easily separate the pair of knob halves 80 when it is desired to remove the knob 26 from the handle body 16. Other forms of mechanical or frictional securements are also contemplated. It will be appreciated that by combining two knob halves 80, one on top of the other, that the attachment protrusions 86 of one of the knob halves 80 will align with the corresponding attachment apertures 88 formed in the other of the spool halves 80. The knob half 80 may be injected molded, for example, or could be 3D printed.

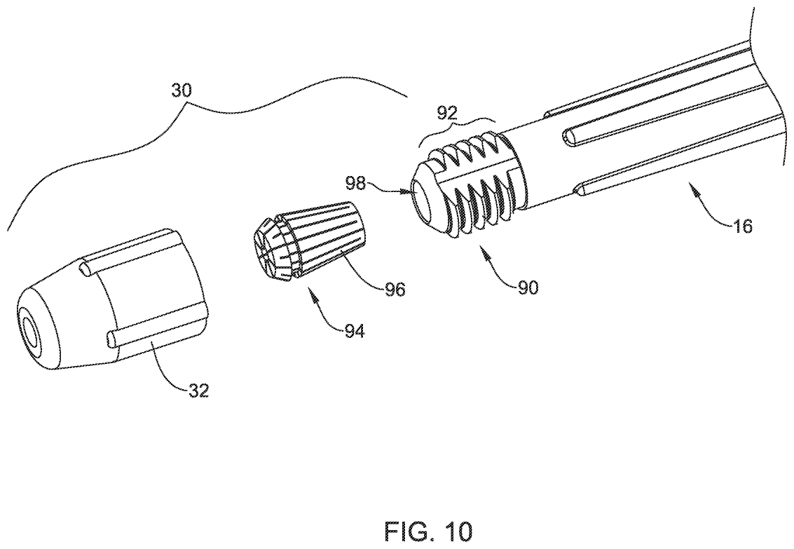

FIG. 10 is an exploded perspective view of the securement region 30. A distal end 90 of the handle body 16 includes a threaded outer surface 92 that is configured to permit the outer collet member 32 to be threadedly engaged with the distal end 90 of the handle body 16. The outer collet member 32 cooperates with an inner collet member 94 to secure the shaft 14 to the handle assembly 12. The inner collet member 94 includes a tapered body 96 that fits into an aperture 98 that extends through the distal end 90 of the handle body 16. As the outer collet member 32 is threaded onto the threaded outer surface 92 of the distal end 90 of the handle body 16, the inner collet member 94 is compressed into the shaft 14, thereby securing the shaft 14. It will be appreciated that the outer collet member 32 may be easily secured onto the distal end 90 of the handle body 16, and that the inner collet member 94 and the outer collet member 32 may be easily removed from the handle body 16 in order to be able to easily remove the handle assembly 12 from the shaft 14.

The flexibility-enhancing patterns formed within the core member 36, as well as other portions of the shaft 14, may include slots. In some embodiments, at least some, if not all of the slots are disposed at the same or a similar angle with respect to a longitudinal axis. The slots can be disposed at an angle that is perpendicular, or substantially perpendicular, and/or can be characterized as being disposed in a plane that is normal to the longitudinal axis. However, in other embodiments, the slots can be disposed at an angle that is not perpendicular, and/or can be characterized as being disposed in a plane that is not normal to the longitudinal axis. Additionally, a group of one or more slots may be disposed at different angles relative to another group of one or more slots. The distribution and/or configuration of the slots can also include, to the extent applicable, any of those disclosed in U.S. Pat. Publication No. US 2004/0181174, the entire disclosure of which is herein incorporated by reference.

The slots may be provided to enhance the flexibility while still allowing for suitable torque transmission characteristics. The slots may be formed such that one or more rings and/or tube segments interconnected by one or more segments and/or beams that are formed, and such tube segments and beams may include portions that remain after the slots are formed. Such an interconnected structure may act to maintain a relatively high degree of torsional stiffness, while maintaining a desired level of lateral flexibility. In some embodiments, some adjacent slots can be formed such that they include portions that overlap with each other about the circumference of the member in which the slots are formed. In other embodiments, some adjacent slots can be disposed such that they do not necessarily overlap with each other, but are disposed in a pattern that provides the desired degree of lateral flexibility.

Additionally, the slots can be arranged along the length of, or about the circumference of, the member including the slots to achieve desired properties. For example, adjacent slots, or groups of slots, can be arranged in a symmetrical pattern, such as being disposed essentially equally on opposite sides, or can be rotated by an angle relative to each other about the axis of the member. Additionally, adjacent slots, or groups of slots, may be equally spaced along the length of the member, or can be arranged in an increasing or decreasing density pattern, or can be arranged in a non-symmetric or irregular pattern. Other characteristics, such as slot size, slot shape, and/or slot angle with respect to the longitudinal axis of the member, can also be varied along the length of the member in order to vary the flexibility or other properties. In other embodiments, moreover, it is contemplated that the portions of the member, such as a proximal section, or a distal section, or the entire member, may not include any such slots.

As suggested herein, slots may be formed in groups of two, three, four, five, or more slots, which may be located at substantially the same location along the axis of the member. Alternatively, a single slot may be disposed at some or all of these locations. Within the groups of slots, there may be included slots that are equal in size (e.g., span the same circumferential distance around the member). In some of these as well as other embodiments, at least some slots in a group are unequal in size (e.g., span a different circumferential distance around the member). Longitudinally adjacent groups of slots may have the same or different configurations. For example, some embodiments may include slots that are equal in size in a first group and then unequally sized in an adjacent group. It can be appreciated that in groups that have two slots that are equal in size and are symmetrically disposed around the tube circumference, the centroid of the pair of beams (e.g., the portion of the member remaining after slots are formed therein) is coincident with the central axis of the member. Conversely, in groups that have two slots that are unequal in size and whose centroids are directly opposed on the tube circumference, the centroid of the pair of beams can be offset from the central axis of the member. Some embodiments of the member include only slot groups with centroids that are coincident with the central axis of the member, only slot groups with centroids that are offset from the central axis of the member, or slot groups with centroids that are coincident with the central axis of the member in a first group and offset from the central axis of the member in another group. The amount of offset may vary depending on the depth (or length) of slots and can include other suitable distances.

Slots can be formed by methods such as micro-machining, saw-cutting (e.g., using a diamond grit embedded semiconductor dicing blade), electron discharge machining, grinding, milling, casting, molding, chemically etching or treating, or other known methods, and the like. In some such embodiments, the structure of a member is formed by cutting and/or removing portions of a tube to form slots. Some example embodiments of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing the shaft 14 may include forming slots using these or other manufacturing steps.

In at least some embodiments, slots may be formed in a member using a laser cutting process. The laser cutting process may include a suitable laser and/or laser cutting apparatus. For example, the laser cutting process may utilize a fiber laser. Utilizing processes like laser cutting may be desirable for a number of reasons. For example, laser cutting processes may allow cutting a number of different cutting patterns in a precisely controlled manner. This may include variations in the slot width, ring width, beam height and/or width, etc. Furthermore, changes to the cutting pattern can be made without the need to replace the cutting instrument (e.g., blade). This may also allow smaller tubes (e.g., having a smaller outer diameter) to be used without being limited by a minimum cutting blade size.

The materials that can be used for the various components of the elongate steerable medical device 10 and the various members disclosed herein may include those commonly associated with medical devices. Components of the elongate steerable medical device 10 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the shaft 14 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the shaft 14 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the shaft 14 to achieve the same result.

In some embodiments, a degree of Magnetic Resonance Imaging (MRI) compatibility is imparted into the shaft 14. For example, portions of the shaft 14 may be made of a material that does not substantially distort the image and create substantial artifacts (e.g., gaps in the image). Certain ferromagnetic materials, for example, may not be suitable because they may create artifacts in an MRI image. Portions of the shaft 14 may be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Components of the shaft 14 may be made of the same material along its length, or in some embodiments, can include portions or sections made of different materials. In some embodiments, the material used to construct components of the shaft 14 may be chosen to impart varying flexibility and stiffness characteristics to different portions of the shaft 14. For example, a proximal section and a distal section may be formed of different materials, for example, materials having different moduli of elasticity, resulting in a difference in flexibility. In some embodiments, the material used to construct a proximal section can be relatively stiff for pushability and torqueability, and the material used to construct a distal section can be relatively flexible by comparison for better lateral trackability and steerability. For example, a proximal section can be formed of straightened 304v stainless steel wire or ribbon and a distal section can be formed of a straightened super elastic or linear elastic alloy, for example a nickel-titanium alloy wire or ribbon.

In embodiments where different portions of the shaft 14 are made of different materials, the different portions can be connected using a suitable connecting technique and/or with a connector. For example, the different portions may be connected using welding (including laser welding), soldering, brazing, adhesive, or the like, or combinations thereof. These techniques can be utilized regardless of whether or not a connector is utilized. The connector may include a structure generally suitable for connecting portions of a guidewire. One example of a suitable structure includes a structure such as a hypotube or a coiled wire which has an inside diameter sized appropriately to receive and connect to the ends of the proximal portion and the distal portion. Other suitable configurations and/or structures can be utilized for connector 26 including those connectors described in U.S. Pat. Nos. 6,918,882 and 7,071,197 and/or in U.S. Patent Pub. No. 2006-0122537, the entire disclosures of which are herein incorporated by reference.

A sheath or covering (not shown) may be disposed over portions or all of the elongate shaft 14 that may define a generally smooth outer surface. In other embodiments, however, such a sheath or covering may be absent from a portion of all of the shaft 14. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the shaft 14 may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over a portion of the shaft 14. Alternatively, the sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinyl alcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An elongate steerable medical device, comprising:
a shaft comprising:
an elongate outer member; and
an elongate inner member slidingly disposed within the elongate outer member; and
a handle assembly removeably securable to the shaft, the handle assembly comprising:
a handle body including a proximal handle;
a spool slidingly and releasably securable to the handle body, the spool adapted to be releasable securable to the elongate inner member;
a knob that is rotatably and releasably securable to the handle body, the knob adapted to be releasably securable to the elongate inner member such that rotation of the knob relative to the handle body causes rotation of the elongate inner member;
a collet removably securable to a distal end of the handle body, the collet adapted to releasably secure the elongate outer member relative to the collet;
wherein movement of the spool relative to the handle body causes the elongate inner member to move relative to the elongate outer member.

2. The elongate steerable medical device of claim 1, wherein the elongate inner member comprises:
an elongate core wire:
a proximal portion that extends proximally from the elongate outer member; and
an asymmetric distal portion having a semi-circular cross-sectional profile; and
a sheath extending over the elongate core wire;
wherein the asymmetric distal portion is secured to the sheath.

3. The elongate steerable medical device of claim 2, wherein the sheath comprises one or more polymeric layers extending over the elongate core wire.

4. The elongate steerable medical device of claim 2, wherein the sheath further comprises a braided layer.

5. The elongate steerable medical device of claim 2, wherein at least a portion of the sheath comprises a laser cut hypotube.

6. The elongate steerable medical device of claim 2, wherein the sheath comprises a distal sleeve that is disposed over and secured to the asymmetric distal portion of the elongate core wire.

7. The elongate steerable medical device of claim 2, wherein the sheath comprises a proximal sleeve disposed over the proximal portion of the elongate core wire.

8. The elongate steerable medical device of claim 2, wherein the asymmetric distal portion of the elongate core wire comprises a flexibility-enhancing pattern formed therein.

9. The elongate steerable medical device of claim 1, wherein the spool comprises a first spool half and a second spool half that are adapted to releasably snap together to slidingly secure the spool to the handle body with the proximal portion of the elongate inner member extending therethrough.

10. The elongate steerable medical device of claim 9, wherein the first spool half and the second spool half are adapted to separate from each other and the handle body in order to allow release of the proximal portion of the elongate inner member.

11. The elongate steerable medical device of claim 1, wherein the elongate inner member further comprises a crimp sleeve secured to the proximal portion of the elongate inner member.

12. The elongate steerable medical device of claim 11, wherein the spool defines an interior cavity adapted to accommodate the crimp sleeve therein in order to secure the proximal portion of the elongate inner member relative to the spool.

13. The elongate steerable medical device of claim 1, wherein the knob comprises a first knob half and a second knob half that are adapted to releasably snap together to rotatably secure the knob to the handle body with the proximal portion of the elongate inner member extending therethrough.

14. The elongate steerable medical device of claim 13, wherein the first knob half and the second knob half are adapted to separate from each other and the handle body in order to allow release of the proximal portion of the elongate inner member from the handle assembly.

15. The elongate steerable medical device of claim 1, wherein the distal end of the handle body includes a threaded outer surface and a cavity defined by the threaded outer surface.

16. The elongate steerable medical device of claim 15, wherein the collet comprises:
a tapered inner collet section disposed within the cavity; and
an outer collet section threadedly engaged with the threaded outer surface and thus securing the elongate outer member relative to the tapered inner collet section.

17. The elongate steerable medical device of claim 1, wherein the shaft comprises a guidewire.

18. A handle assembly adapted for use with a steerable elongate medical device, the steerable elongate medical device including an elongate outer member and an elongate inner member disposed within the elongate outer member, the handle assembly comprising:
a handle body including a proximal handle;
a first spool half and a second spool half that are adapted to releasably snap together to form a spool slidingly secured to the handle body, the spool adapted to releasable secure the elongate inner member;
a collet removably securable to a distal end of the handle body, the collet adapted to releasably secure the elongate outer member device;
wherein steering the steerable elongate medical device is enabled by moving the spool relative to the handle body.

19. The handle assembly of claim 18, further comprising a knob including a first knob half and a second knob half that are adapted to releasably snap together, the knob rotatably secured to the handle body with the elongate inner member extending therethrough such that rotation of the knob relative to the handle body causes rotation of the elongate inner member.

20. A steerable guidewire assembly, comprising:
a steerable guidewire comprising:
an elongate outer member;
an elongate inner member slidingly disposed within the elongate outer member, the elongate inner member including:
a proximal portion that extends proximally from the elongate outer sheath;
an asymmetric distal portion having a semi-circular cross-sectional profile; and
a sheath extending over the elongate inner member, the asymmetric distal portion secured to the sheath;
a handle assembly removeably securable to the steerable guidewire, the handle assembly comprising:
a handle body including a proximal handle;
a spool slidingly securable to the handle body, the spool adapted to enable translation of the elongate inner member;
a knob rotatably securable to the handle body, the knob adapted to enable rotation of the elongate inner member;
a collet removably securable to a distal end of the handle body, the collet adapted to releasably secure the elongate outer member relative to the collet;
wherein relative movement of the spool and/or the knob facilitates steering of the steerable guidewire.
